(19) United States
(12) Patent Application Publication
(10) Pub. No.: US 2014/0378772 A1
(43) Pub. Date: Dec. 25, 2014

(54) IMPLANTING ORGAN PORTS

(71) Applicant: Mayo Foundation for Medical Education and Research, Rochester, MN (US)

(72) Inventors: Thoralf M. Sundt, III, Boston, MA (US); Brent R. Phillips, Waukesha, WI (US)

(21) Appl. No.: 14/341,534

(22) Filed: Jul. 25, 2014

Related U.S. Application Data
(63) Continuation of application No. 13/388,336, filed on Feb. 1, 2012, now abandoned.
(60) Provisional application No. 61/231,942, filed on Aug. 6, 2009.

(51) Int. Cl. A61B 17/34

(52) U.S. Cl. CPC A61B 17/3423; A61B 2017/3425; A61B 2017/00252
USPC 600/204

(57) ABSTRACT

This document relates to medical devices (e.g., organ ports such as transapical heart ports) and methods and materials for implanting and using such medical devices. For example, organ ports and entry devices that can be used to implant an organ port are provided.

### CROSS-REFERENCE TO RELATED APPLICATION

This application is a continuation of U.S. application Ser. No. 13/388,336, filed Feb. 1, 2012, which is a National Stage application under 35 U.S.C. §371 of International Application No. PCT/US2010/044429, filed Aug. 4, 2010, which claims priority to U.S. Provisional Application Ser. No. 61/231,942, filed on Aug. 6, 2009, the contents of which are herein incorporated by reference in their entirety.

### BACKGROUND

1. Technical Field

This document relates to medical devices (e.g., organ ports such as transapical heart ports) and methods and materials for implanting and using such medical devices. For example, this document provides entry devices that can be used to implant an organ port.

2. Background Information

Many medical procedures require access to the interior of an organ. For example, cardiac surgical procedures routinely require access to the interior of the heart. In the case of heart surgeries, transapical approaches can allow cardiac surgeons to access the interior of the heart via the apex. Through such access, a surgeon can, for example, replace or repair a mitral or aortic valve or can perform other surgical procedures.

### SUMMARY

This document relates to medical devices (e.g., organ ports such as transapical heart ports) and methods and materials for implanting and using such medical devices. For example, this document provides entry devices that can be used to implant an organ port. Such medical devices and entry devices can be used to provide a surgeon (e.g., a cardiac surgeon) with secure access to the interior of an organ (e.g., a heart). For example, a transapical heart port provided herein can be implanted using an entry device provided herein to allow cardiac surgeons to (1) perform heart surgeries with reduced tissue trauma and a reduced access site size, (2) induce less stress to the heart during initial access, (3) pass instruments in and out of the access site with ease, (4) maintain hemostasis particularly while passing instruments in and out of the access site, (5) gain access without the need of sutures, which can allow re-access to the heart without a risk of tearing sutures, and (6) obviate the need to remove the transapical heart port or repair the apex of the heart after the procedure (e.g., the transapical heart port may remain in place for an extended period of time or permanently).

In general, one aspect of this document features a system for implanting an organ port into a mammal. The system comprises, or consists essentially of, (a) an entry device comprising a body and a blade attached to the body, and (b) an organ port comprising a housing having a first end and a second end, wherein the housing defines a channel extending between the first end and the second end, wherein the first end is configured to be inserted into an organ of the mammal, wherein the channel is configured to provide repeated access to the interior of the organ through the channel, wherein the first end comprises a securing portion configured to secure the first end to an interior region of the organ, and wherein the second end comprises a securing portion configured to secure the second end to an exterior region of the organ, and wherein the entry device is configured to be withdrawn through the channel. The blade can be in a spiral configuration around the body. The body can comprise two or more blades. The body can comprise three or more blades. The body can comprise a distal end and a proximal end, and wherein the blade is located at or towards the distal end. The body can taper to a narrower diameter in a direction from the proximal end to the distal end. The body can define a lumen that extends from the distal end to the proximal end, the being adapted to receive a guide wire. The blade can extend in a direction from the proximal end to the distal end. The organ port can comprise a hemostatic valve attached to the housing and located within the channel. The organ can be a heart. The organ port can comprise a hemostatic valve attached to the housing and located within the channel, and wherein the hemostatic valve can be configured to reduce blood loss from the heart through the channel. The organ port can comprise pliable material that allows flexion of the port during beating of the heart. The size of the channel can allow passage of a prosthetic heart valve. The securing portion of the first end or the securing portion of the second end can comprise hooks configured to be embedded within the myocardium of the heart. The hooks can comprise a shape memory alloy for self-embedding within the myocardium upon deployment of the transapical heart port into the heart. The securing portion of the first end and the securing portion of the second end can comprise hooks configured to be embedded within the myocardium of the heart. The hooks can comprise a shape memory alloy for self-embedding within the myocardium upon deployment of the transapical heart port into the heart. The securing portion of the first end and/or the securing portion of the second end can comprise a balloon filled with a filler material that conforms to the contours of the heart. The filler material can be a liquid or gas. The filler material can harden over time. The filler material can remain pliable to allow flexion of the transapical heart port during beating of the heart. The balloon can comprise a donut shape configured about the first end or the second end. The balloon can comprise multiple flanges configured about the first end or the second end. The surface of the balloon can comprise a coating that promotes endothelization or cell growth. The securing portion of the first end can comprise a first balloon, and the securing portion of the second end can comprise a second balloon larger than the first balloon. The first and second balloons can be filled with a filler material that conforms to the contours of the heart. The second end can comprise multiple access points that allow multiple concurrent accesses. The organ port can comprise a plug located within the channel and configured to provide permanent hemostasis. The plug can be secured to the channel using threads, snaps, hooks, or an adhesive. The plug can define a chamber configured to deliver drugs to the heart. The chamber can comprise an access site for refilling the chamber. The transapical heart port can comprise a sheath that provides access to the second end through the chest wall covering the heart. The sheath can be detachable from the transapical heart port. The sheath can comprise a hemostatic valve. The sheath can comprise two or more hemostatic valves. The sheath can define a balloon filler channel. The organ port can comprise two or more hemostatic valves attached to the housing and located within the channel.

In another aspect, this document features a method for accessing the interior of a organ in a mammal. The method comprises, or consist essentially of, (a) contacting an exterior of the organ with an entry device comprising a body and a blade attached to the body to form an opening into the interior, and (b) inserting an organ port into the opening under condition wherein the organ port forms a channel from the exterior to the interior. The method can comprise withdrawing the entry device from the mammal. The withdrawing step can comprise removing the entry device from the interior to the exterior through the channel. The organ port can comprise a housing having a first end and a second end, wherein the housing defines the channel extending between the first end and the second end, wherein the first end is configured to be inserted into an organ of the mammal, wherein the channel is configured to provide repeated access to the interior of the organ through the channel, wherein the first end comprises a securing portion configured to secure the first end to an interior region of the organ, and wherein the second end comprises a securing portion configured to secure the second end to an exterior region of the organ.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a cross-sectional view showing an example of a transapical heart port in a heart.

FIGS. 2A-2C are cross-sectional views showing examples of securing transapical heart ports in a heart.

FIGS. 3A-3B are top views showing examples of balloons for securing transapical heart ports.

FIG. 4 is a cross-sectional view showing an example of multiple access locations in a transapical heart port.

FIG. 5 is a cross-sectional view showing an example of a transapical heart port including a plug that provides long term hemostasis.

FIGS. 6A-6B are cross-sectional views showing examples of transapical heart ports having an attached sheath that provides access to the transapical heart port.

FIG. 7 is a flow chart showing an example of a process for accessing the interior of a heart.

FIG. 8 is a flow chart showing an example of a process for accessing the interior of a heart.

FIG. 9 is a flow chart showing an example of a process for accessing and performing a therapeutic treatment through a previously placed transapical heart port.

FIG. 10 is a flow chart showing an example of a process for placing a transapical heart port using an imaging probe.

FIG. 11 is a flow chart showing another example of a process for placing a transapical heart probe.

FIGS. 12A-B are diagrams of exemplary imaging probes and needles for use in a system for accessing the interior of a heart.

FIG. 13 is a diagram of an exemplary imaging probe and needle deployed in the interior of a heart.

FIG. 14 is a diagram of a system for accessing the interior of a heart.

FIGS. 15A-E are diagrams of embodiments of an entry device that is part of a system for accessing the interior of a heart.

FIGS. 16A-E are diagrams depicting the use of an embodiment of a system used for accessing the interior of a heart.

FIGS. 17A-H are diagrams of several embodiments of plugs and port bodies belonging to a system for accessing the interior of a heart.

FIGS. 18A-H are diagrams of embodiments of a system used for accessing the interior of a heart.

FIGS. 19A-D are diagrams of an alternative entry device of a system for accessing the interior of a heart.

FIGS. 20A-D are diagrams of an alternative entry device of a system for accessing the interior of a heart.

FIGS. 21A-D are diagrams of an alternative entry device of a system for accessing the interior of a heart.

FIG. 22 is a photograph of a transapical heart port implanted into a pig heart with a blood pressure reading of 104/69.

FIG. 23 is a photograph of a transapical heart port implanted into a pig heart with a blood pressure reading of 209/148.

FIG. 24 is a photograph of a transapical heart port implanted into a pig heart with a blood pressure reading of 198/199.

FIG. 25 is a photograph of a transapical heart port implanted into a pig heart with a blood pressure reading of 321/229.

Like reference symbols in the various drawings indicate like elements.

### DETAILED DESCRIPTION

This document relates to medical devices. For example, this document provides organ ports (e.g., transapical heart ports), entry devices that can aid in the implantation of an organ port, methods for making organ ports (e.g., transapical heart ports), methods for making entry devices, systems that include an organ port and an entry device, and methods for using an organ port and entry device. The organ ports provided herein can be inserted into any type of organ (e.g., a heart, stomach, bladder, bowel, vessels, abdominal cavity, lung, uterus, and vaginal canal. For example, a transapical heart ports provided herein can be inserted and secured to the apex of a beating heart to provide secure access to the inside or interior of the heart. The devices provided herein can have one or more one-way (or hemostatic) valves such that access to the inside of an organ (e.g., a heart via the apex) is provided without blood loss around the instruments being introduced into the organ (e.g., a heart). In the case of a transapical heart port provided herein, the port can be used during surgeries where the patient's heart remains beating. The ports provided herein can be used for inserting instruments of various types into the organ. For example, valves, catheters, suture devices, and repair devices can be inserted into a heart via a transapical heart port provided herein.

The ports provided herein can be self-securing to the organ. For example, a transapical heart port provided herein can include a self-securing mechanism (e.g., a sutureless securing mechanism). In some cases, a transapical heart port provided herein can remain in place after completion of the operation being performed on the heart. The ports provided herein can have a deployment mechanism such as a dilator system over a wire. For example, a method such as the Seldinger technique used in cardiac catheterization labs can be used to deploy a transapical heart port. In some cases, a transapical heart port provided herein can be inserted at the apex of the heart, for example, using an open surgical incision or percutaneously. In some cases, a transapical heart port itself can provide secure access such that instruments can be exchanged during the intracavitary surgery without concern that one would lose control of the apex of the heart (e.g., to prevent bleeding through or around the transapical heart port and to maintain blood pressure in the patient).

In some cases, an entry device provided herein can be used to introduce an organ port into an organ. Such an entry device can include blades that can cut into the tissue of the desired organ. For example, an entry device provided herein can include a body having one or more blades. Such blades can include a forward facing cutting edge such that the forward facing cutting edge contacts the organ's tissue as the entry device is advanced in a forward direction. In some cases, the blades can have a spiral configuration. In general, an entry device can be configured to cut through a wall of an organ to create a channel for an organ port. The organ port can be attached behind the entry device as the entry device is advanced during the cutting process. Once a channel is formed across the organ's wall and the entry port is in position, the entry device can be withdrawn through the organ port and removed from the patient. In some cases, the entry port can define a lumen (e.g., a central lumen) such that a guide wire can be to guide the advancement of the entry device.

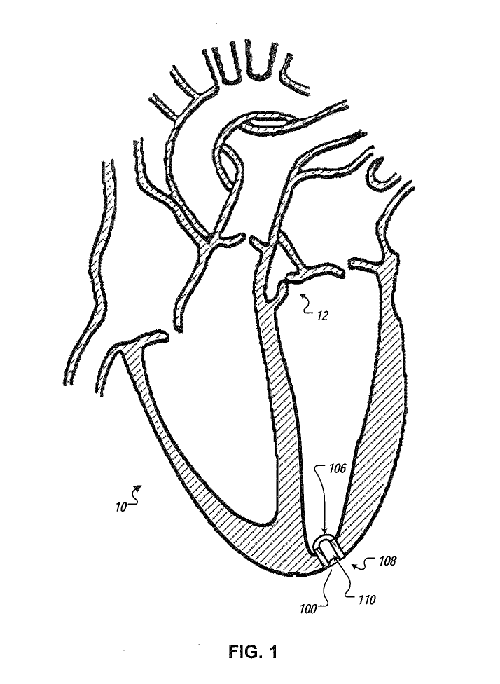

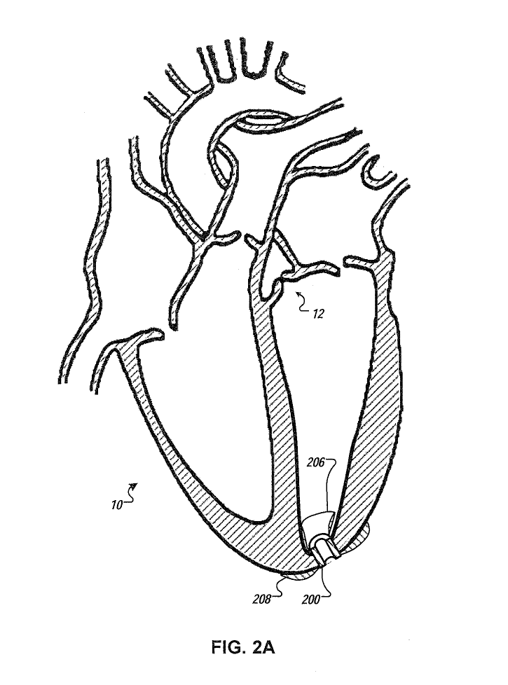

FIG. 1 is a cross-sectional view showing an example of a transapical heart port 100 in a heart 10. The transapical heart port 100 includes a housing having a first end 106. The first end 106 is inserted into the heart 10 at the apex of the heart 10. While shown here as being inserted in the left ventricle of the heart 10, the transapical heart port 100 can also be inserted into the right ventricle of the heart 10. The housing of the transapical heart port 100 also includes a second end 108. The housing with first end 106 and the second end 108 defines a channel 110. The channel 110 provides access to the interior of the heart 10. For example, a drug can be delivered to the interior of the heart 10 through the channel 110. In some cases, a heart valve 12 can be repaired or replaced via access through the channel 110.

The transapical heart port 100 can be made of various materials, such as metals, plastics, and polymers. In some cases, the transapical heart port 100 can be made of a material that is pliable. The pliable material may allow flexion of the transapical heart port 100 during beating of the heart 10. The flexion of the material in the transapical heart port 100 can prevent or reduce tissue damage to the heart 10 and dislodging of the transapical heart port 100 from the heart 10.

In some cases, the channel 110 has an interior diameter that is sufficiently large to allow passage of a prosthetic heart valve through the channel 110. For example, the channel 110 may have a diameter of five or more millimeters (e.g., at least five, six, seven, eight, nine, ten, eleven, twelve, 13, 14, 15, 16, 17, 18, 19, or 20 mm). In some cases, the diameter can be between about 10 mm and about 12 mm. A prosthetic heart valve may be used, for example, in replacing the heart valve 12.

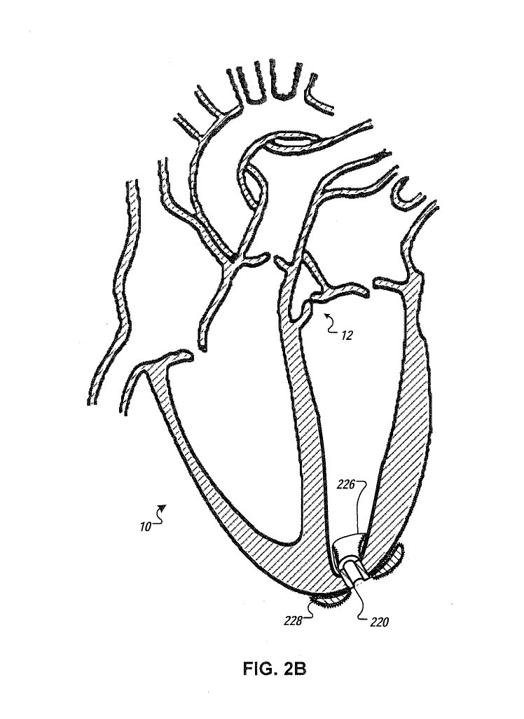

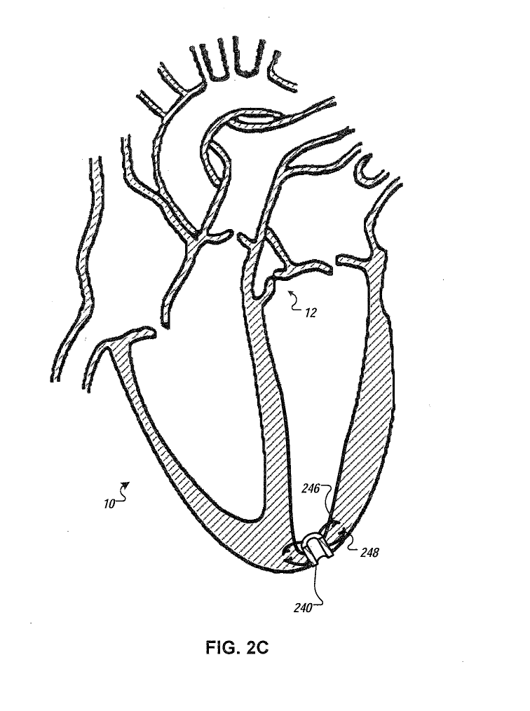

FIGS. 2A-2C are cross-sectional views showing examples of securing transapical heart ports in a heart. In some cases, the method used to secure the transapical heart port provides a pull-out strength with a pressure of between about 300 and about 400 mm of mercury (mmHg). For example, the method used to secure the transapical heart port can provide a pull-out strength with a pressure of at least 300 mmHg FIG. 2A shows a transapical heart port 200 in a heart 10. The transapical heart port 200 is secured at the interior of the heart 10 by one or more interior balloons 206. The transapical heart port 200 is also secured at the exterior of the heart 10 by one or more exterior balloons 208. In some cases, the exterior balloons 208 cover a greater surface area of the heart 10 than the interior balloons 206. The balloons can be made using biomedical balloon material.

The interior balloons 206 and the exterior balloons 208 can secure the transapical heart port 200 to the heart 10 by conforming to the anatomy of the heart 10. For example, the balloons can conform to the curvature of the interior and exterior heart walls as well as thickness of the heart wall at the apex of the heart 10.

In some cases, the interior balloons 206 and/or the exterior balloons 208 are filled or inflated with a gas (e.g., carbon dioxide) or a liquid (e.g., saline). In some case, the material used to fill the balloons can harden over time. For example, a polymer such as acrylate, foam, or gel can be used to fill the balloons. In some cases, the hardness of the filler material remains pliable to allow flexion during beating of the heart 10.

FIG. 2B shows a transapical heart port 220 in a heart 10. The transapical heart port 220 is secured at the interior of the heart 10 by one or more interior balloons 226. The transapical heart port 220 is secured at the exterior of the heart 10 by one or more exterior balloons 228. In some cases, the interior balloons 226 and/or the exterior balloons 228 can have a surface that promotes cell growth or endothelization. The surface also can limit thrombolytic potential. In some cases, the surface can further secure the transapical heart port 220 to the heart 10. For example, the transapical heart port 220 and/or the balloons can be coated with a material that promotes cell growth. In another example, the transapical heart port 220 and/or the balloons can have a surface texture that promotes cell growth and/or further secures the transapical heart port 220 to the heart 10. In some cases, the balloons can be made of (or coated) with a fabric, such as Dacron. In some cases, the balloons can be coated with a cell growth compound such as collagen, fibrin, or another cell growth promoting biomatrix.

FIG. 2C shows a transapical heart port 240 in a heart 10. The transapical heart port 240 can include one or more interior hooks 246 and one or more exterior hooks 248 that secure the transapical heart port 240 to the heart 10. Particularly, the hooks can embed within the myocardium of the heart 10. In some cases, the tips of the hooks have barbs that hold the hooks in the myocardium. In some cases, the hooks are deployed by an active process, such as by actuating a hinge of a hook. In some cases, the hooks self-embed within the myocardium. For example, the hooks can include a shape memory alloy, such as nickel titanium or nitinol. Deploying the hooks in the heart 10 can warm the shape memory alloy material in the hooks, causing the hooks to reshape or bend and embed within the myocardium of the heart 10.

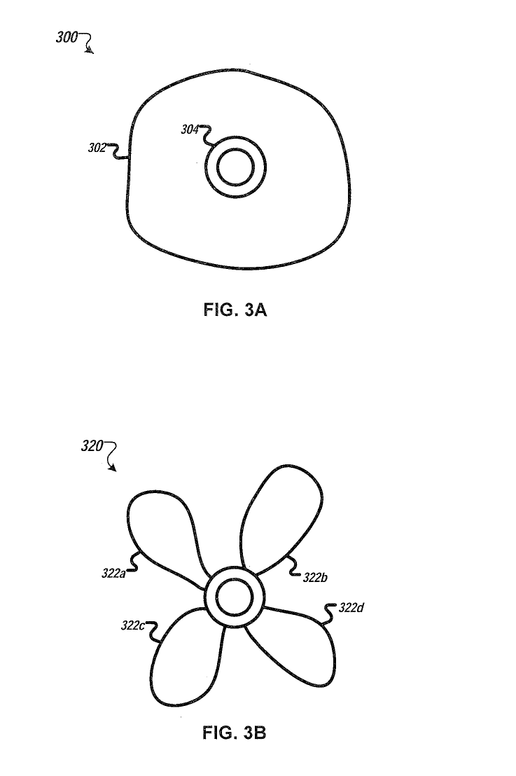

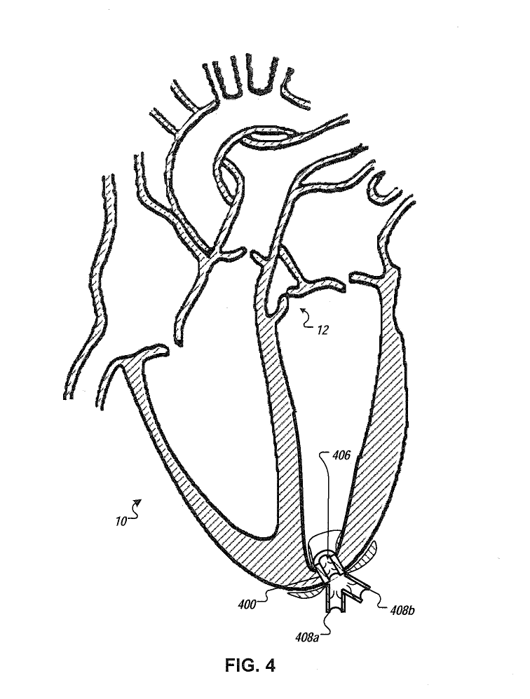

FIGS. 3A-3B are top views showing examples of balloons for securing transapical heart ports. FIG. 3A shows a circular balloon 302. The circular balloon 302 forms a round or doughnut shape about the opening 304 of a transapical heart port 300. FIG. 3B shows multiple flange balloons 322a-d. The flange balloons 322a-d can be distributed about the opening of a transapical heart port 320. The circular balloon 302 and the flange balloons 322a-d can act to secure a transapical heart port (such as the ports 300 and 320) to a heart as described herein.

FIG. 4 is a cross-sectional view showing an example of multiple access locations in a transapical heart port 400. The transapical heart port 400 is inserted in a heart 10. The transapical heart port 400 includes one or more hemostatic valves 406. The hemostatic valves can prevent or reduce blood loss from the heart 10. For example, the hemostatic valves 406 can include one-way valves and/or a self-sealing septum as is described in U.S. Pat. No. 5,718,682, filed on Jun. 28, 1996, by Elton M. Tucker, and entitled “Access port device and method of manufacture.”

The transapical heart port 400 also includes multiple access points 408a-b. The access points 408a-b can provide for multiple concurrent accesses to the interior of the heart 10 through the transapical heart port 400. For example, multiple instruments can be inserted into the heart 10 simultaneously through the transapical heart port 400 using the access points 408a-b. In some cases, the access points 408a-b include one or more hemostatic valves that prevent or reduce blood loss from the heart 10.

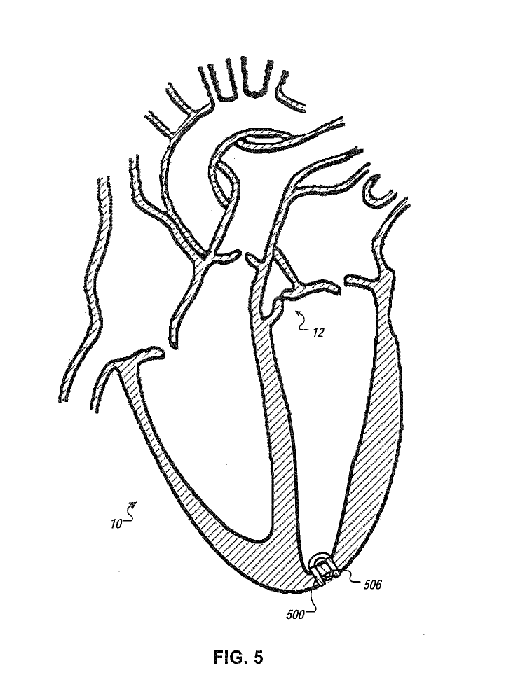

FIG. 5 is a cross-sectional view showing an example of a transapical heart port 500 in a heart 10. The transapical heart port 500 can include a plug 506 that provides long term or permanent hemostasis. The plug 506 can be left in place within the transapical heart port 500 over an extended period of time. The plug 506 can engage the transapical heart port 500 using, for example, snaps, hooks, adhesive (e.g., glue), or other securing methods. In some cases, the plug 506 can be removed for subsequent procedures that access the interior of the heart 10. In one example, a system can employ threads within the transapical heart port 500 and around the plug 506. A tool can be used to screw and unscrew the plug 506. In some cases, the plug 506 can have a pull-out strength with a pressure between about 500 and about 600 mmHg. For example, the plug 506 can have a pull-out strength with a pressure greater than about 300 mmHg. In some cases, the plug 506 can include a chamber for delivering one or more drugs to the interior of the heart 10. Such drugs can be released over time. In some cases, the plug 506 can include an access site for refilling the drug chamber.

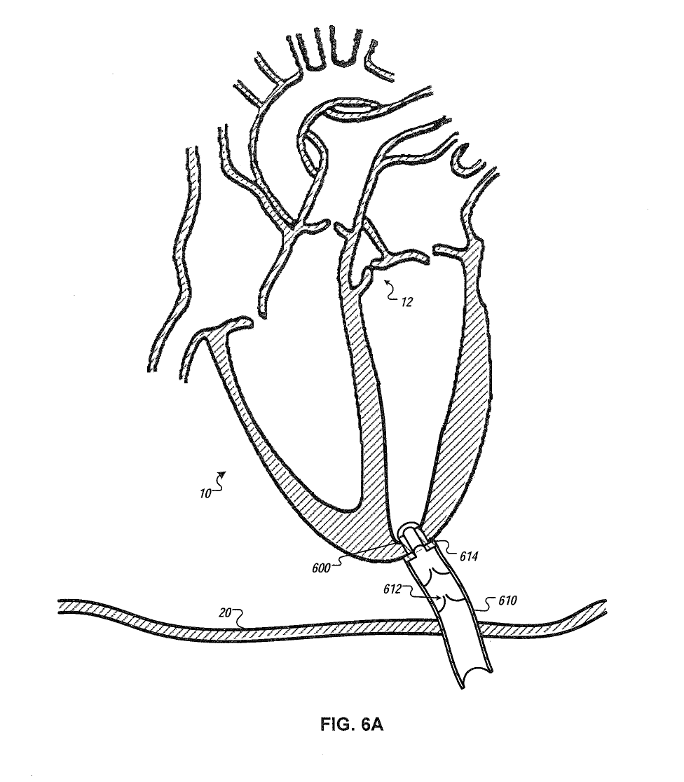

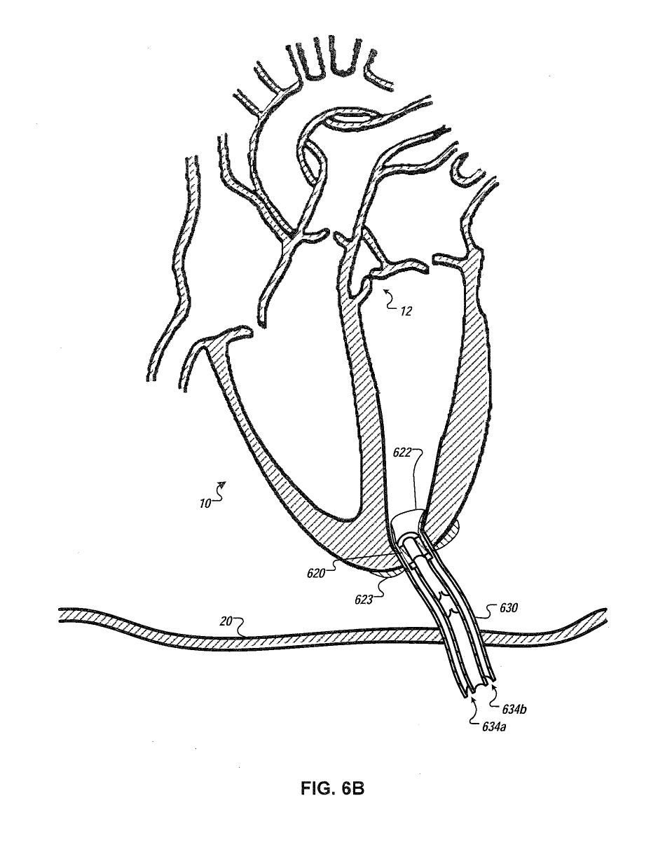

FIG. 6A is a cross-sectional view showing an example of a transapical heart port 600 in a heart 10. Attached to the transapical heart port 600 is a sheath 600 that provides access to the transapical heart port 600 through a chest wall 20 covering the heart 10. In some cases, the sheath 600 can include one or more hemostatic valves 612 that prevent or reduce blood loss from the heart 10. The sheath 600 can provide access to the transapical heart port 600 (and correspondingly the interior of the heart 10) from outside the body through the chest wall 20. While described herein as passing through the “chest wall,” the sheath 600 can pass through an alternate part of the body, such as the abdomen or neck. In some cases, the sheath 600 can prevent or reduce the need for large surgical incisions and/or damage to intervening tissue that results from passing surgical instruments through the body to the transapical heart port 600. In some cases, the sheath 600 can be detached from the transapical heart port 600 after completion of a procedure that accesses the interior of the heart 10. In some cases, a plug, such as the plug 506 described with respect to FIG. 5, can be passed and installed in the transapical heart port 600 through the sheath 600.

The sheath 600 can include an attachment device 614, such as a threaded screw, clips, or luer locks. The sheath 600 can be attached to the transapical heart port 600 prior to inserting the transapical heart port 600 into the heart 10 or after inserting the transapical heart port 600 into the heart 10. The sheath 600 can be detached from the transapical heart port 600 after performing a procedure and a sheath can be reattached for another procedure at a later time.

FIG. 6B is a cross-sectional view showing an example of a transapical heart port 620 in a heart 624. Attached to the transapical heart port 620 is a sheath 630 that provides access to the transapical heart port 620 through a chest wall 20. The transapical heart port 620 can be secured to the heart 624 by one or more interior balloons 622 and one or more exterior balloons 623. The sheath 630 can include an inner wall and an outer wall that define one or more inflation channels 636a-b. The inflation channels 636a-b can provide access to the balloons for filling the balloons with a gas or liquid. In some cases, a plug, such as the plug 506 described with respect to FIG. 5, can be inserted into the transapical heart port 620 through the sheath 630. The plug can be used to sever and seal the inflation ports that connect the sheath 630 to the balloons.

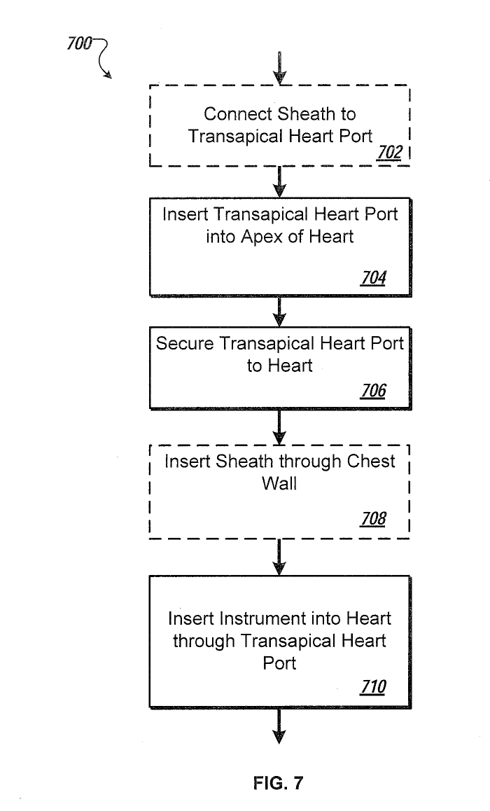

FIG. 7 is a flow chart showing an example of a process 700 for accessing the interior of a heart. The process 700 can be performed, for example, by a device such as the transapical heart ports 100, 200, 220, 240, 300, 320, 400, 500, 600, and 620. For clarity of presentation, the description that follows uses the transapical heart ports 100, 200, 220, 240, 300, 320, 400, 500, 600, and 620 as the basis of an example for describing the process 700. However, another device, or combination of devices, can be used to perform the process 700. Optionally, the process 700 begins with connecting (702) a sheath to a transapical heart port. For example, the sheath 630 can be connected to the transapical heart port 620. The process 700 inserts (704) the transapical heart port into the apex of a heart. For example, the transapical heart port 100 can be inserted into the apex of the heart 10 through an incision or percutaneously. A ventricle, such as the left ventricle, can be accessed with a wire (e.g., small theracotomy).

In some implementations, the Seldinger technique may be used to insert the transapical heart port into the apex of the heart. A needle is inserted into the apex of the heart. A guide wire can be advanced through the needle and into the interior of the heart. The needle is then removed, and the guide wire is left in place. The guide wire is used to insert a dialator/introducer system. The transapical heart port (and optionally the sheath) can be inserted on the introducer system over the wire. After the transapical heart port is inserted, the guide wire and introducer system can be removed.

The process 700 secures (706) the transapical heart port to the heart. For example, the transapical heart ports 300 and 320 can be secured using balloons while the transapical heart port 342 can be secured using hooks. In some cases, a combination of interior and/or exterior securing devices can be used. In some cases, a combination of balloons and hooks can be used. In some implementations, the balloons attached to the transapical heart port 620 can be filled from outside the body through the inflation channels 636a-b in the sheath 630.

Optionally, the process 700 inserts (708) a sheath through a chest wall that covers the heart. For example, the sheath 630 can be inserted through the chest wall 20.

The process 700 inserts (710) an instrument into the heart through the transapical heart port. For example, a prosthetic valve or a plug that delivers a drug to the heart can be inserted through the transapical heart port 620. In some cases, the insertion can occur from outside the body through the sheath 630. In some implementations, the sheath can be detached from the transapical heart port after the procedure. In some implementations, the transapical heart port remains in place after removal of the sheath. In some implementations, inserting the plug into the transapical heart port detaches the sheath from the transapical heart port. The connection between the sheath and the transapical heart port can be made using threads (e.g., screwing a portion of the sheath onto or into a portion of the transapical heart port), clips, luer locks, etc.

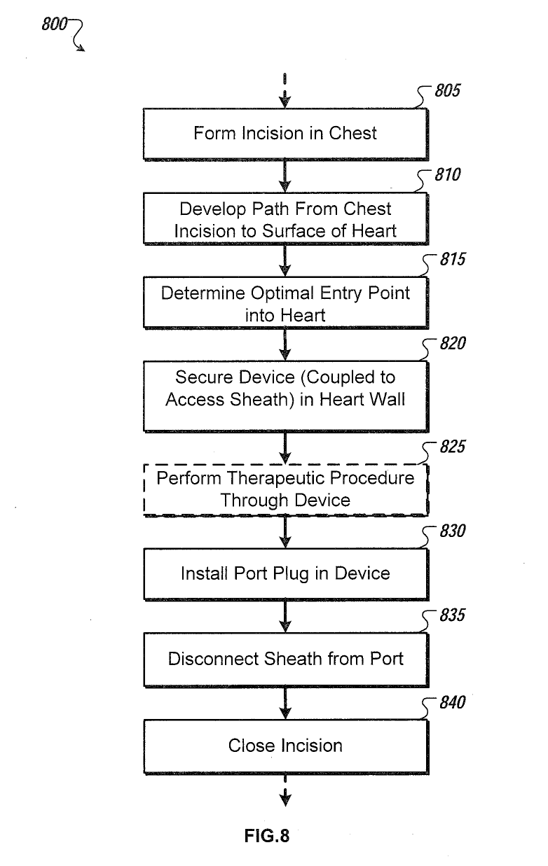

Referring now to FIG. 8, in some embodiments, a surgeon or other trained medical professional can implement the process 800 for using a transapical heart port (e.g., the heart ports 100, 200, 220, 240, 300, 320, 400, 500, 600, and 620) to access the interior of a heart. In operation 805, a small incision can be formed in the chest of a patient. In operation 810, a path can be developed from the incision formed in operation 805, through intervening tissue, to the surface of the heart (e.g., near the apex). In operation 815, the optimal point of entry from the surface of the heart to the interior can be determined using, for example, an ultrasonic imaging probe. For example, an imaging probe can be used to determine where along the apex of the heart to enter. Exemplary anatomical features that can be assessed can include the location of the intraventricular septum, the location of the papillary muscles of the left ventricle, the thickness of the wall of the apex of the left ventricle, the location of blood vessels within the wall of the heart, near the apex. Identification of the optimal point of entry is described in more detail below in connection with FIG. 12

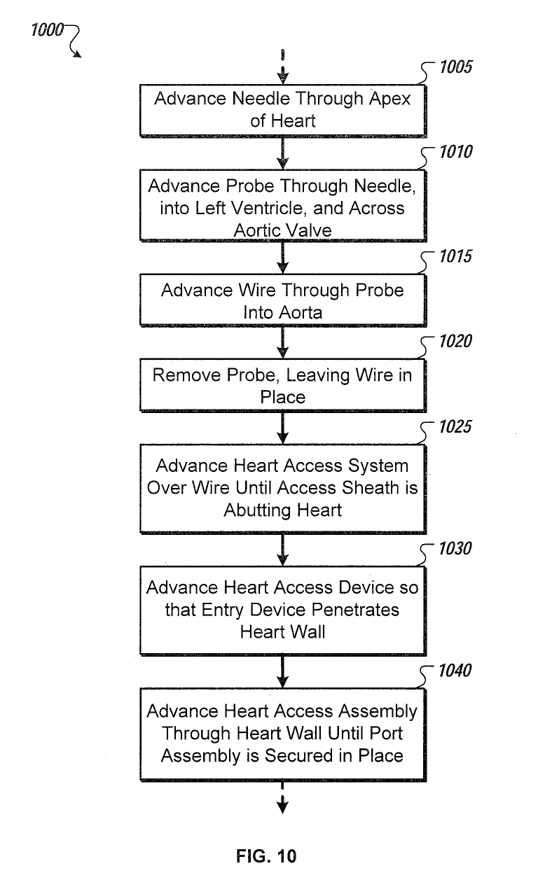

In operation 820, an entry device, such as a transapical heart port, can be secured in the wall of the heart in the point of entry as determined in operation 815. Furthermore, the entry device can be coupled to the distal end of an access sheath for better access to the entry device. The entry device can be coupled to the sheath such that a relatively fluid-tight connection is formed between the entry port and the access sheath, thus fluidly connecting the lumens of the entry device and the access sheath. Furthermore, the access sheath can be configured (e.g., with one or more valves) to reduce or eliminate the loss of fluid (e.g., blood from the interior of the heart) through the proximal end of the sheath. For example, the entry device can allow cardiac surgeons to (1) perform heart surgeries with reduced tissue trauma and a reduced access site size, (2) induce less stress to the heart during initial access, (3) promptly pass instruments in and out of the access site, (4) maintain hemostasis particularly while passing instruments in and out of the access site, (5) gain access without the need of sutures, which can allow re-access to the heart without a risk of tearing sutures, and (6) obviate the need to remove the transapical heart port or repair the apex of the heart after the procedure (e.g., the transapical heart port may remain in place for an extended period of time or permanently). The placement and securing of transapical heart ports is described in more detail below in connection with FIGS. 10-11, 15A-C.

In operation 825, one or more therapeutic procedures can be optionally performed using devices that can be passed through the access sheath and port device and into the heart. Examples of such procedures include, without limitation, valve repair procedures, valve replacement procedures, biopsy procedures, tissue removal (myectomy) procedures, repair of ventricular septal defect procedures, and procedures for delivering cells for cellular therapy or vectors for gene therapy. Examples of devices that can be passed through a device provided herein include, without limitation, catheters (e.g., ablation catheters, artificial valve delivery catheters, and suture delivery catheters), imaging devices (e.g., ultrasound and/or visual imaging devices), biopsy/tissue removal devices (e.g., a biotome), and needles.

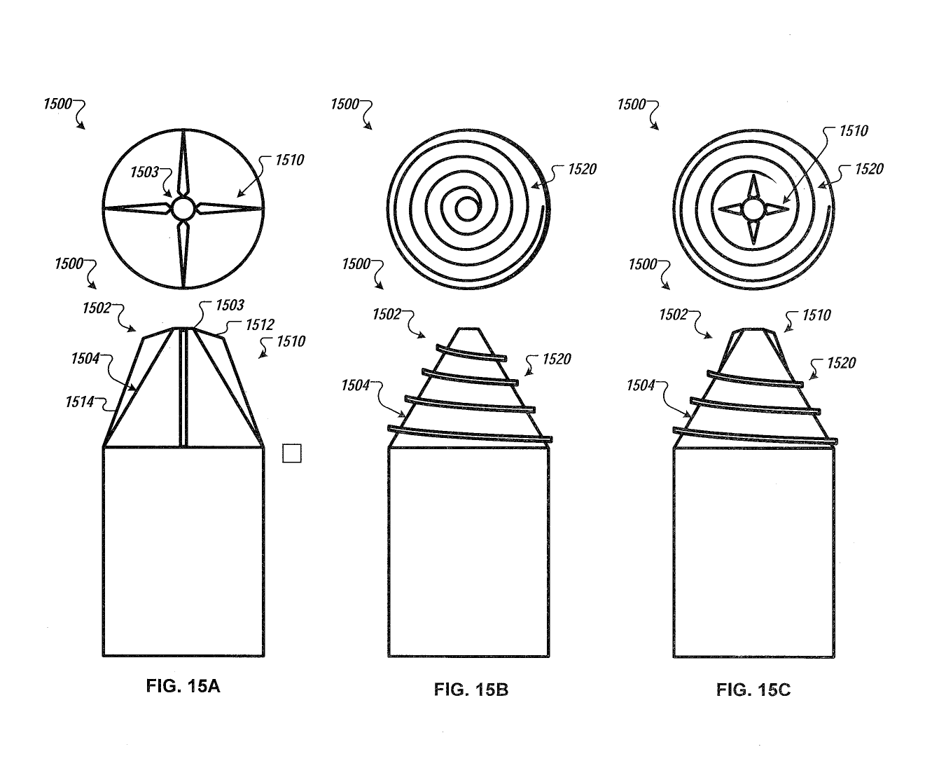

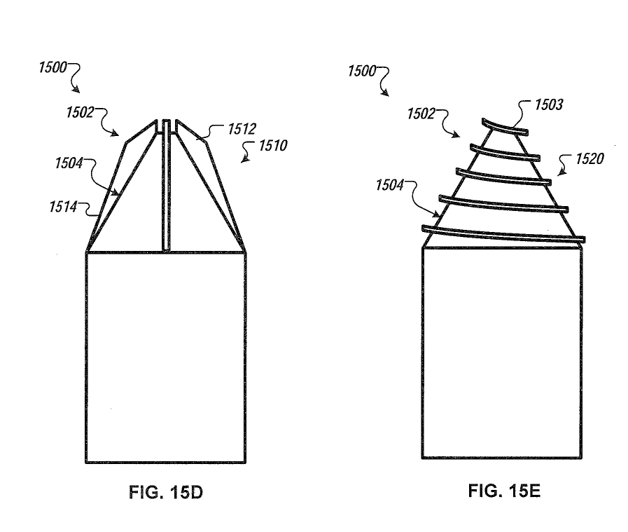

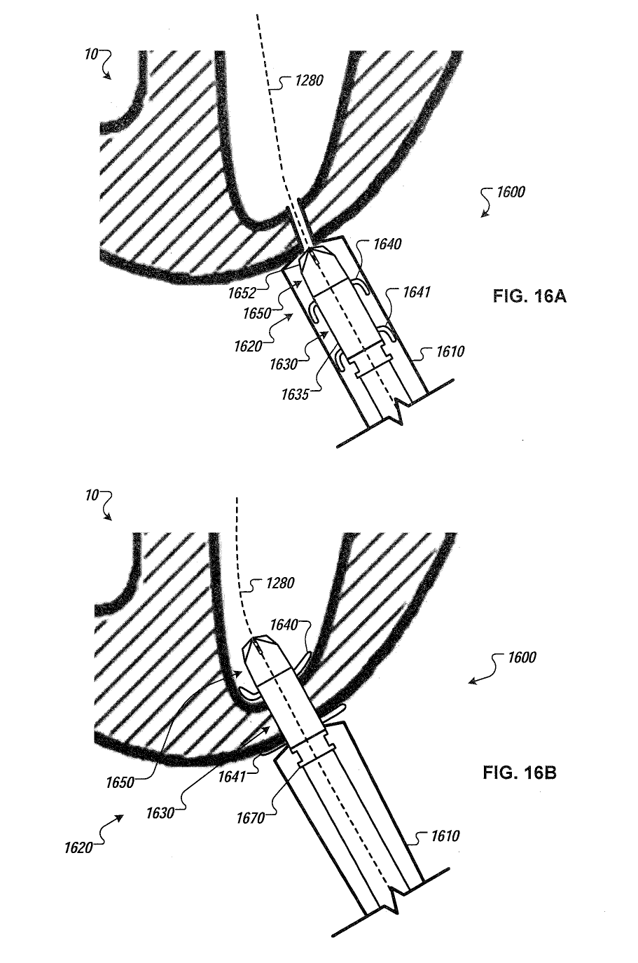

In operation 830, the transapical heart port can be reversibly plugged. As described below in more detail in connection with FIGS. 15D-E, a port plug can be advanced through the access sheath into the lumen of the transapical port. Here, the port plug can be reversibly coupled to the port to maintain homeostasis and can be later removed to gain access to the interior of the heart. The plug can engage the port via one or more of threads, hooks, snaps and the like. In operation 835, the sheath can be removed from the port, for example, by unscrewing the sheath from the transapical port. In operation 840, the incision formed during operation 805 can be closed.

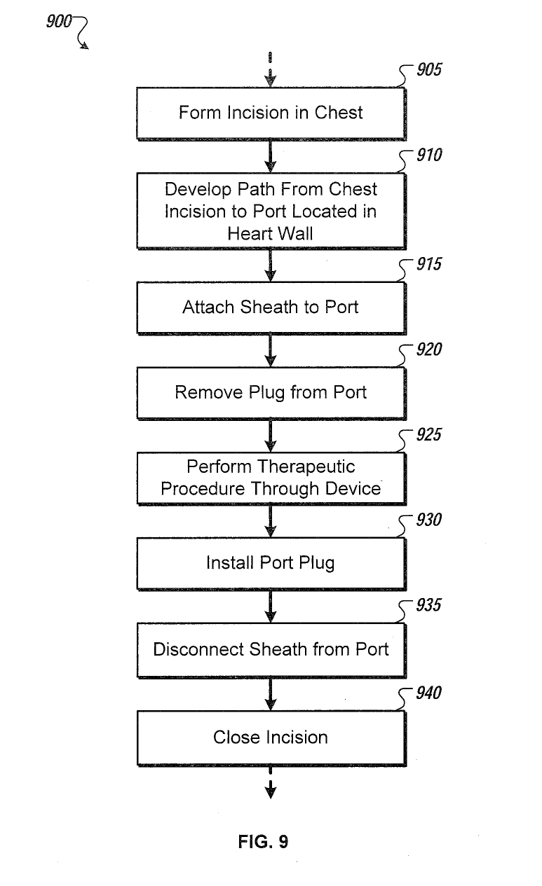

Referring now to FIG. 9, in some embodiments, a surgeon or other trained medical professional can implement the process 900 for accessing the interior of a heart in which an entry device (e.g., the heart ports 100, 200, 220, 240, 300, 320, 400, 500, 600, and 620) has been previously placed. In operation 905, a small incision can be formed in the chest of a patient. In operation 910, a path can be developed from the incision formed in operation 905, through intervening tissue, to a point on the surface of the heart where a transapical port has been previously placed. In operation 915, an access sheath can be advanced through the incision and developed path until the distal end of the access sheath engages the previously placed transapical port, thus allowing for a substantially fluid-tight connection between the heart, the port, and the access sheath. In operation 920, a plug engaged in the lumen of the port can be removed, creating a fluid connection between the interior of the access sheath and the interior of the heart. Features of the access sheath (e.g., valves) can reduce or eliminate the loss of blood from the interior of the heart through the proximal end of the access sheath. In this manner, devices can be advanced through the proximal end of the access sheath, through the transapical port, and into the interior of the heart.

In operation 925, one or more therapeutic procedures can be optionally performed using devices that can be passed through the access sheath and port device and into the heart. Examples of such procedures include, without limitation, valve repair procedures, valve replacement procedures, biopsy procedures, tissue removal (myectomy) procedures, repair of ventricular septal defect procedures, and procedures for delivering cells for cellular therapy or vectors for gene therapy. Examples of devices that can be passed through a device provided herein include, without limitation, catheters (e.g., ablation catheters, artificial valve delivery catheters, and suture delivery catheters), imaging devices (e.g., ultrasound and/or visual imaging devices), biopsy/tissue removal devices (e.g., a biotome), and needles.

In operation 930, the transapical heart port can be reversibly plugged. As described below in more detail in connection with FIGS. 15D-E, a port plug can be advanced through the access sheath into the lumen of the transapical port. Here, the port plug can be reversibly coupled to the port to maintain homeostasis and can be later removed to gain access to the interior of the heart. The plug can engage the port via one or more of threads, hooks, snaps and the like. In operation 935, the access sheath can be removed from the port, for example, by unscrewing the sheath from the transapical port. In operation 940, the incision formed during operation 905 can be closed.

Referring now to FIG. 10, a surgeon or other trained medical professional can perform a process 1000 for securing a heart entry device in the heart wall of a patient. In some examples, the process 1000 can be performed as part of a process for accessing the interior of a heart, such as described in connection with FIG. 8. In operation 1005, a needle can be advanced through the wall of the heart (e.g., in the wall of the left ventricle) into the interior of the heart. In operation 1010, an imaging probe (e.g., an ultrasound imaging probe) can be advanced through the lumen of the needle into the interior of the heart. Images obtained from the imaging probe can be used to advance the probe (e.g., configured to include steerability) to the aortic valve, across the aortic valve, and into the aorta. The probe can include proximal and distal entry holes fluidly connecting an interior lumen of the probe to the exterior of the probe. An exemplary probe is described in more detail in connection with FIG. 12B. In operation 1015, a wire can be passed through the entry hole near the proximal end of the probe, through the lumen, out of the hole near the distal end of the probe, and into the aorta. Once the distal end of the wire is positioned within the aorta, the probe can be removed in operation 1000, leaving the wire in place.

In operation 1005, a heart access device and placement system, including an entry device (e.g., the transapical heart ports 100, 200, 220, 240, 300, 320, 400, 500, 600, 620, and the like) can be placed over the wire, such that the proximal end of the wire is inserted through a lumen of the entry device. As described in more detail in connection with FIG. 15A, the entry device can be contained at least partially within the interior of an access sheath (e.g., to protect the entry device, to maintain fixation members in a non-deployed state, to facilitate advancement of the entry device through the chest of the patient, to minimize damage to surrounding tissue, and the like). The system can be advanced until the distal end of the sheath is in contact with the wall of the heart and the opening of the sheath surrounds the location in the heart wall through which the wire is located.

In operation 1030, the system can be further advanced, while the sheath remains abutting the heart, such that an entry device penetrates the hole in the heart wall through which the wire is located. Features of the device, such as a beveled distal portion, a cutting surface near the distal end, and the like (described in more detail below) can facilitate entry into the heart wall by, for example, expanding the original hole in the heart wall without causing uncontrolled tearing. The entry device can be further advanced in operation 1035, until fixation members of the port assembly transition from the non-deployed to the deployed state, thus securing the port assembly within the wall of the heart. This is described in more detail below in connection with FIG. 15B.

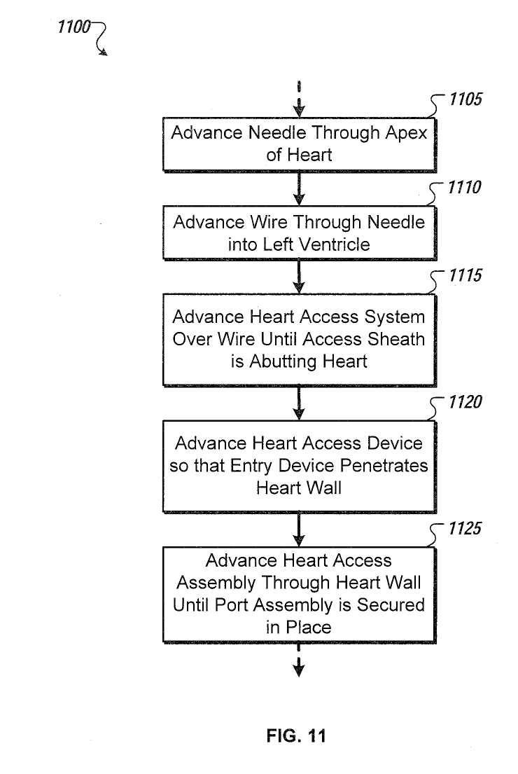

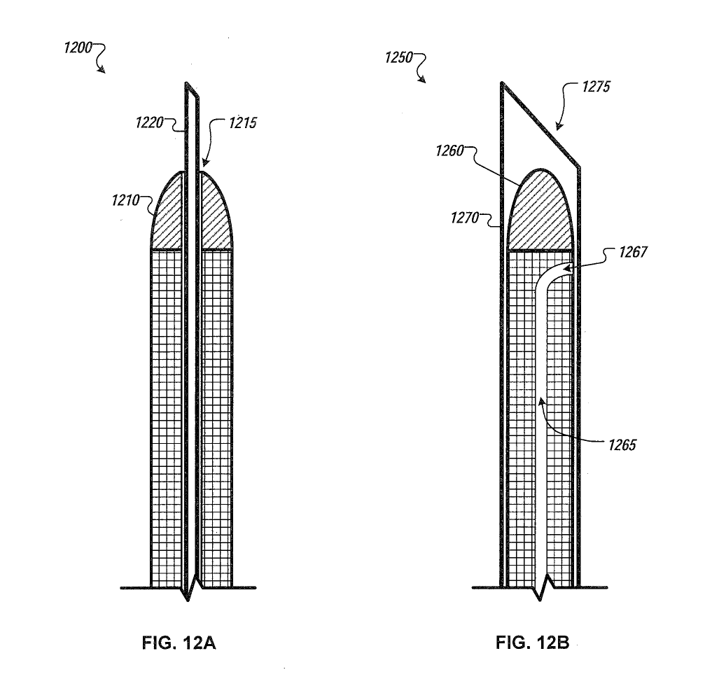

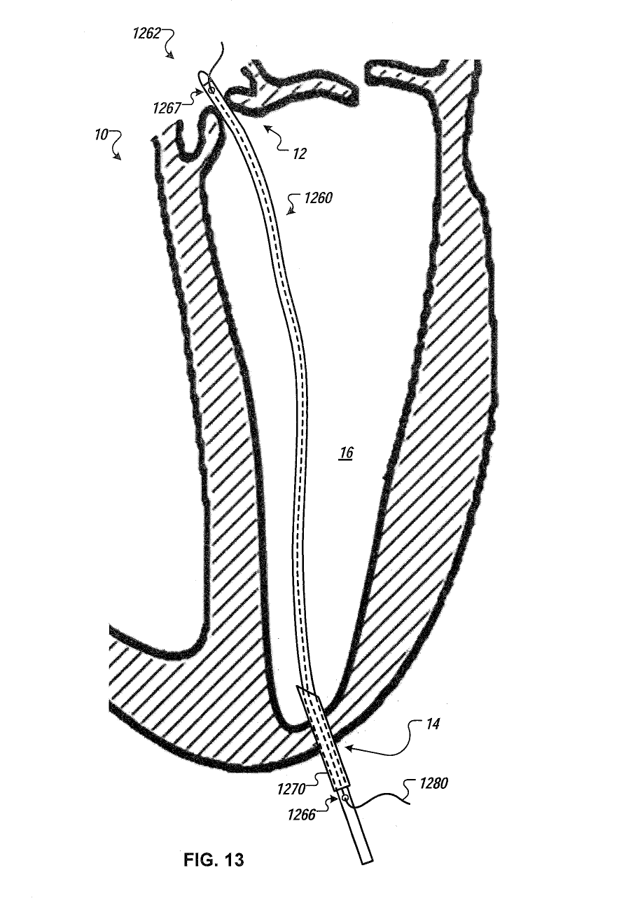

Referring now to FIG. 11, a surgeon or other trained medical professional can perform a process 1100 for securing a heart entry device in the heart wall of a patient.

In some examples, the process 1100 can be performed as part of a process for accessing the interior of a heart, such as described in connection with FIG. 8. In operation 1105, a needle can be advanced through the wall of the heart (e.g., in the wall of the left ventricle) into the interior of the heart. In operation 1110, a wire can be passed through the lumen of the needle and into the interior of the heart.

In operation 120, a heart access device and placement system, including an entry device (e.g., the transapical heart ports 100, 200, 220, 240, 300, 320, 400, 500, 600, 620, and the like) can be placed over the wire, such that the proximal end of the wire is inserted through a lumen of the entry device. As described in more detail in connection with FIG. 15A, the entry device can be contained at least partially within the interior of an access sheath (e.g., to protect the entry device, to maintain fixation members in a non-deployed state, to facilitate advancement of the entry device through the chest of the patient, to minimize damage to surrounding tissue, and the like). The system can be advanced until the distal end of the sheath is in contact with the wall of the heart and the opening of the sheath surrounds the location in the heart wall through which the wire is located.

In operation 125, the system can be further advanced, while the sheath remains abutting the heart, such that an entry device penetrates the hole in the heart wall through which the wire is located. Features of the device, such as a beveled distal portion, a cutting surface near the distal end, and the like (described in more detail below) can facilitate entry into the heart wall by, for example, expanding the original hole in the heart wall without causing uncontrolled tearing. The entry device can be further advanced in operation 1130, until fixation members of the port assembly transition from the non-deployed to the deployed state, thus securing the port assembly within the wall of the heart. This is described in more detail below in connection with FIG. 15B.

Referring now to FIGS. 12A-B, a combination imaging probe and entry needle can be used to identify an optimal point of entry into the heart, place the distal portion of a guide wire in the interior of a heart, and the like. In some embodiments, the imaging probe can be an ultrasound imaging probe. FIG. 12A depicts a needle/imaging probe assembly 1200 that includes an imaging probe 1210 with a central lumen 1215 through which a hollow needle 1220 can be passed. In this example, the needle 1220 can be passed down the lumen 1215 and into a heart after an optimal entry point into the heart has been determined using the imaging probe 1210. FIG. 12B depicts an assembly 1250 that includes an imaging probe 1260 that can be advanced within a lumen 1275 of a needle 1270. The needle 1270 can be advanced into the heart (over the probe 1260) after an optimal entry point into the heart has been determined using the imaging probe 1260. The imaging probe 1260 can include a central lumen 1265, through which a guide wire can be passed. The wire can, for example, enter the central lumen 1265 through a proximal opening (not shown), advance through the central lumen 1265, and exit the central lumen through a distal opening 1267.

Referring now to FIG. 13, the assembly 1250 can be used to optimize the placement of a wire into the aorta of heart. For example, an imaging probe (such as the imaging probe 1260) can be used to find an entry point 14 in a heart 10. The needle 1270 can be advanced through the wall of the heart 10 (e.g., by puncturing the cardiac tissue) at the entry point 14. Once the distal portion of the needle 1260 is located within the left ventricle 16, the imaging probe 1260 can be advanced through the needle 1270 until the distal portion 1262 of the imaging probe 1260 is located within the left ventricle 16 (not shown). The imaging probe 1260 can be further advanced and steered until it passes through the heart valve 12 into the aorta. The imaging probe 1260 can include standard steering (e.g., wires to control tip direction and the like) to control the direction in which the probe 1260 moves and can be guided using images captured by the imaging probe 1260. The imaging probe 1260 can accept a wire 1280 through a proximal opening 1266. The wire 1280 can be advanced through the lumen 1265 (see FIG. 12B) and exit through the distal opening 1267. Once the wire 1280 is positioned in a desired location (e.g., the distal portion is located within the aorta), the probe 1260 can be removed, leaving the wire in the desired location.

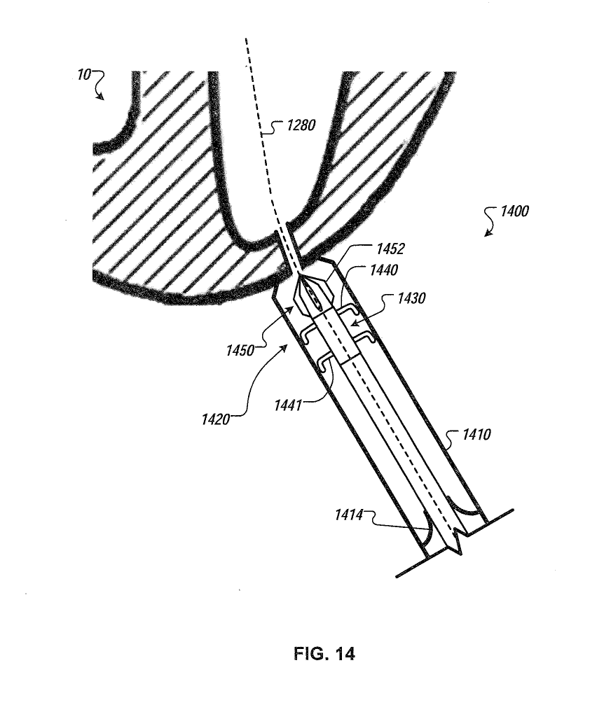

Referring now to FIG. 14, in some embodiments, a heart access device and placement system 1400 can be used access the interior of a heart (such as the heart 10). In some embodiments, the system 1400 can include an access/delivery sheath 1410 for delivering a heart access assembly 1420 to the surface of a heart. The heart access assembly can include a transapical port assembly 1430 that can be positioned in the wall of a heart to allow devices top be passed from the exterior of the heart to the interior of the heart and an entry device 1450 to facilitate placement of the port assembly 1430 by form or enlarging an opening in the heart 10.

In some embodiments, the heart access assembly 1420 is placed over the wire 1280 and pushed to the surface of the heart 10. The heart access assembly 1420 (including the transapical port assembly 1430 and the entry device 1450) can be placed within an access/delivery sheath 1410 to constrain fixation members 1440 and 1441, to protect surrounding tissue from blades 1452 of the entry device 1450, and the like. The access/delivery sheath 1410 can include one or more one-way valves 1414, for example, that can allow tools to pass through the interior of the sheath 1410, but not allow blood to flow out of the proximal end of the sheath 1410 from the heart 10 (e.g., maintain hemostasis). In some embodiments, the sheath 1410 can branch out near the proximal portion (such as depicted in FIG. 4) to allow multiple tools to be passed/used within the heart 10 at the same time. The port assembly 1430, entry device 1440, and the sheath 1410 can include echogenic/fluoroscopic coatings to help visualize the devices during a procedure.

Referring now to FIG. 15A-E, a variety of configurations of cutting surfaces can be included in the distal portion 1502 of an entry device 1500 to facilitate placement of a transapical port (not shown) through the left ventricle muscle wall. In some embodiments, the distal portion 1502 of an entry device 1500 can include one or more beveled edges 1504 to help get the device 1500 through the left ventricle wall without tearing. The distal portion 1502 can include cutting elements to help in placement of a port in the wall of left ventricle. Referring now to FIG. 15A, in some embodiments, a series of straight blades 1510 can be coupled to the exterior surface of an entry device 1500 such that the blades extend radially outward from the surface of the entry device 1500. The blades 1510 can be placed as close to the distal tip opening 1503 of the entry device 1500 as possible. Any number of the blades 1510 (e.g., 0, 1, 4, 7, and the like) can be arranged around the circumference of the entry device 1500. The blades can be configured such that a front edge 1512 and a long edge 1514 of the blade 1510 include cutting surfaces.

Referring now to FIG. 15B, in some embodiments, spiral blades 1520 can be configured in a corkscrew arrangement and spiral down the shaft of the entry device 1500. In this design the operator may apply a rotational force, in addition to or in lieu of, a translational force to advance the entry device 1500 into the wall of a heart. Referring now to FIG. 15C, the entry device 1500 can include a combination of blades, such as the straight cutting blades 1510 and the spiral blades 1520. The straight blades 1510 can assist initial entry in to the heart wall, while the spiral blades 1520 can complete the placement.

Referring now to FIGS. 15D-E, to further facilitate entry of a trans-apical port device into the ventricular muscle, blades (e.g., the blades 1510 and 1520) at the distal tip portion 1502 of an entry device 1500 can be arranged such that they overhang the distal tip opening 1503 of the central lumen (e.g., a wire port). This can provide for a sharp tip at the very distal end of the device which could help with device entry into the muscle of the ventricle. FIG. 15D depicts a straight blade concept and FIG. 15E depicts a spiral blade concept.

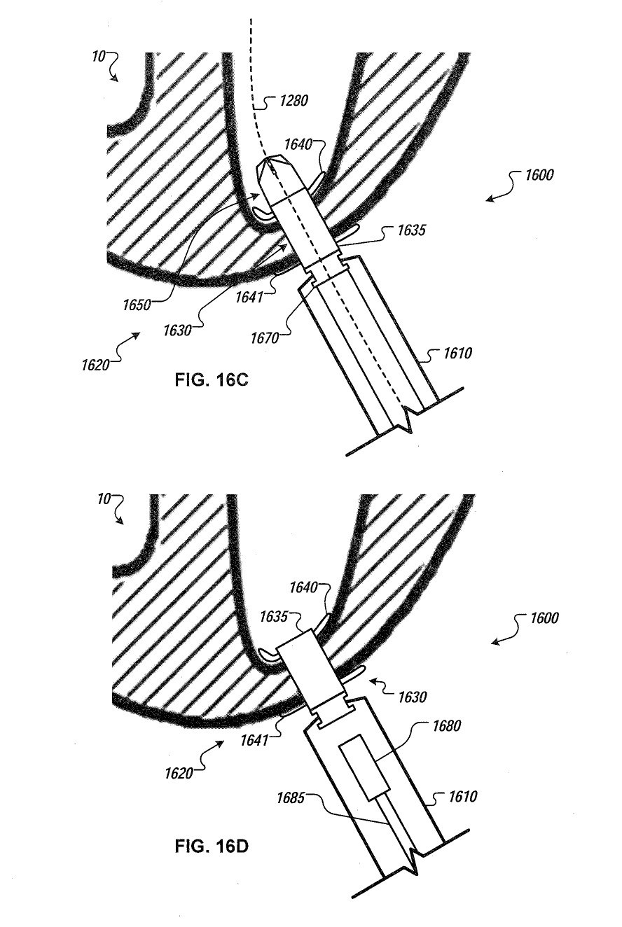

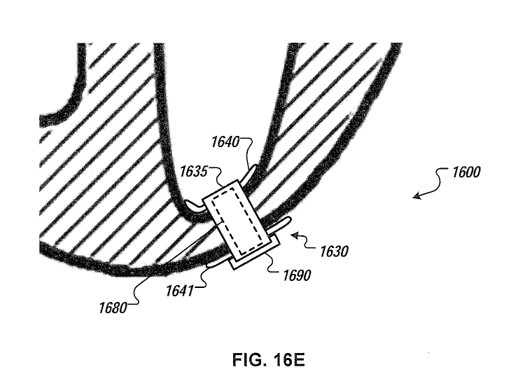

Referring now to FIGS. 16A-E, in some embodiments, a heart access device and placement system 1600 can be used access the interior of a heart (such as the heart 10). In some embodiments, the system 1600 can include an access/delivery sheath 1610 for delivering a heart access assembly 1620 to the surface of a heart. The heart access assembly 1620 can include a transapical port assembly 1630 that can be positioned in the wall of a heart to allow devices top be passed from the exterior of the heart to the interior of the heart and an entry device 1650 to facilitate placement of the port assembly 1630 by form or enlarging an opening in the heart 10. The port assembly 1630 can include an optional port adapter 1670 (described in more detail below) and a plug 1680 (see FIG. 16E) that can be positioned inside a lumen of the port assembly 1630 to restrict the flow of blood from the inside of the heart 10 to the outside. The port assembly 1630 can include a port body 1635 including an internal lumen for passing devices through and fixation members 1640 and 1641 for securing the port assembly 1630 in the wall of a heart.

Referring now to FIG. 16A, in use, the heart access assembly 1620 can be placed over the wire 1280 and pushed to the surface of the heart 10. The heart access assembly 1620 (including the transapical port assembly 1630, the entry device 1650, and the port adaptor 1670) can be placed within an access/delivery sheath 1610 to constrain the fixation members 1640 and 1641, to protect surrounding tissue from blades 1652 of the entry device 1650, and the like. The access/delivery sheath 1610 can include one or more one-way valves (not shown), similar to the valves 1414 depicted in FIG. 14. For example, the one-way valves can allow tools to pass through the interior of the sheath 1610, but not allow blood to flow out of the proximal end of the sheath 1610 from the heart 10 (e.g., maintain hemostasis). In some embodiments, the sheath 1610 can branch out near the proximal portion (such as depicted in FIG. 4) to allow multiple tools to be passed/used within the heart 10 at the same time. The port assembly 1630, entry device 1650, and the sheath 1610 can include echogenic/fluoroscopic coatings to help visualize the devices during a procedure.

Referring now to FIG. 16B, in some embodiments, the access assembly 1620 is advanced into the wall of the heart 10 while the sheath is held in place. As the port assembly 1630 is pushed out of the sheath and into the interior of the heart (e.g., into the left ventricle) the fixation members 1640 and 1641 can expand. As depicted in FIG. 16C, the sheath 1610 can be slid back to engage with the port adaptor 1670 via any engagement means (e.g., hooks, snaps, slots, adhesives, threads, etc.). In some embodiments, flanges 1612 of the sheath 1610 can reversibly engage corresponding notches 1672 in port adapter 1670. The entry device 1650 can be disengaged from the port body 1635 (e.g., by unscrewing, disengaging locking pins, and the like) and removed through the sheath 1610.

Referring now to FIG. 16D, the port plug 1680 can be used to seal the internal lumen of the port body 1635, for example, upon completion of a therapeutic procedure. The plug 1680 can be advanced through the sheath 1610 and the port adapter 1670 to the port assembly 1630. The plug 1680 can be configured with a variety of features (e.g., threads, hooks, pins, and the like) such that the plug 1680 can reversibly engage the port body 1635. In some embodiments, a plug tool 1685 can be used to screw the plug 1680 into the port body 1635 through the sheath 1610 by, for example, engaging at the proximal end of the plug 1680. The port adaptor 1670 can be uncoupled from the port assembly 1630, for example, by unscrewing, releasing an attachment means, or the like. Uncoupling the port adaptor 1670 can also release the sheath 1610 from the port assembly 1630. In some embodiments, the sheath 1610 can be coupled to the port assembly 1630 (e.g., by corresponding threads, attachment pins, and the like) without the use of a port adapter (such as the port assembly 1670). In these embodiments, the sheath 1610 can be uncoupled from the port assembly 1630.

Referring now to FIG. 16E, after the plug 1680 is coupled to the interior of the port body 1635 an optional port cap 1690 can be coupled to the proximal end of the port assembly 1630. In some embodiments, the cap 1690 can be attached by screwing the cap 1690 onto threads originally used to couple the port adapter 1670 to the port assembly 1630. The port cap 1690 can be used as a redundant measure to secure the plug 1680 into the port body 1635, used to provide an atraumatic surface over the port, used to keep debris and tissue out of the internal lumen of the port body 1635, and the like. In some embodiments, the cap can include silicone, or any other soft, compliant material that will not irritate the surrounding tissues.

In some cases, the sheath can be left in place, and the cap can be delivered within sheath. In this case, a cap-port attachment that is separate from the sheath-port attachment system can be used. In some cases, a cap can be delivered using a cap delivery or deployment tool. The tool can use the same channel that the sheath used to get from the exterior of the body to the organ (e.g., heart). The tool can be long and relatively rigid with the cap at the distal end. The cap and tool can be positioned using imaging guidance (ultrasound and/or fluoroscopy). The cap can be deployed onto the port (e.g., by screwing, clipping, gluing, etc.). The tool can then be removed.

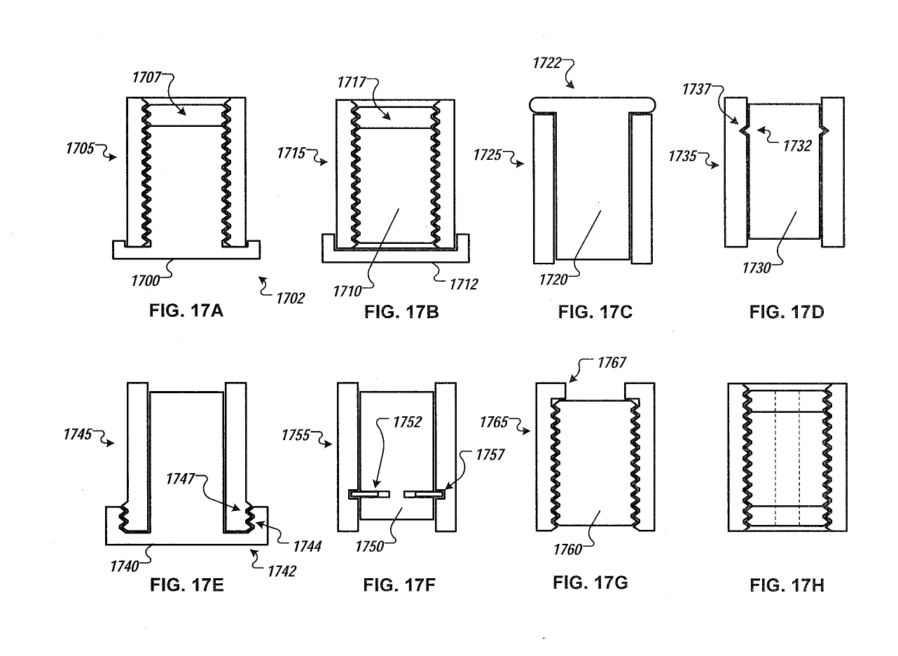

Referring now to FIGS. 17A-H, a variety of configurations can be used to couple a port plug (such as the port plug 1680 shown in FIG. 16E) to a port body. For example, FIG. 17A depicts a port plug assembly 1700 with an integral cap portion 1702 that can be threaded into a port body 1705. The plug assembly 1700 can include echogenic/fluoroscopic markings/coatings 1707 to help determine when the plug assembly 1700 is fully deployed (e.g., flush with the distal surface of the port). FIG. 17B depicts a port plug assembly 1710 and a separate cap 1712 that can be coupled to a port body 1715 (e.g., by threads, snaps, and the like). The plug 1710 can include echogenic/fluoroscopic markings/coatings 1717 to help determine when the plug assembly 1710 is fully deployed (e.g., flush with the distal surface of the port).

Referring now to FIG. 17C, a plug 1720 can include an expandable polymer which can “mushroom” at a distal portion 1722 to secure the plug 1720 in a port body 1725. FIG. 17D depicts a plug 1730 that includes protrusions 1732 can mate with corresponding grooves 1737 in a port body 1735. FIG. 17E depicts a configuration in which a port body 1745 includes threads 1747 on the exterior of the port 1745. A plug 1740 including an integral cap portion 1742 and threads 1744 can be coupled to the port body 1745 by engaging the corresponding threads 1744 and 1747.

Referring now to FIG. 17F, a plug 1750 can include retractable/deployable pins 1752 that can engage the wall of a port body 1755, for example, at corresponding grooves 1757 in a port body 1755. FIG. 17G depicts a configuration in which a plug stop 1767 is built into the wall of a port body 1765 to prevent the plug 1760 from being over-advanced in the port body 1765. FIG. 17H depicts a plug that can contain or be coated with agents to facilitate physiologic outcomes. Also, the internal lumen of the plug could contain an amount of drug that slowly elutes for time either into the heart or into surrounding tissues. Exemplary drugs that could be used and stored in the plug can include anti-inflammatory/steroids (e.g., to limit inflammation and/or adhesions in the pericardial space), anti-thrombolytics (e.g., eluted into the left ventricle from the left ventricle facing surface of the plug), anti-fibrotics (e.g., to prevent adhesion on the pericardial surface), and the like.

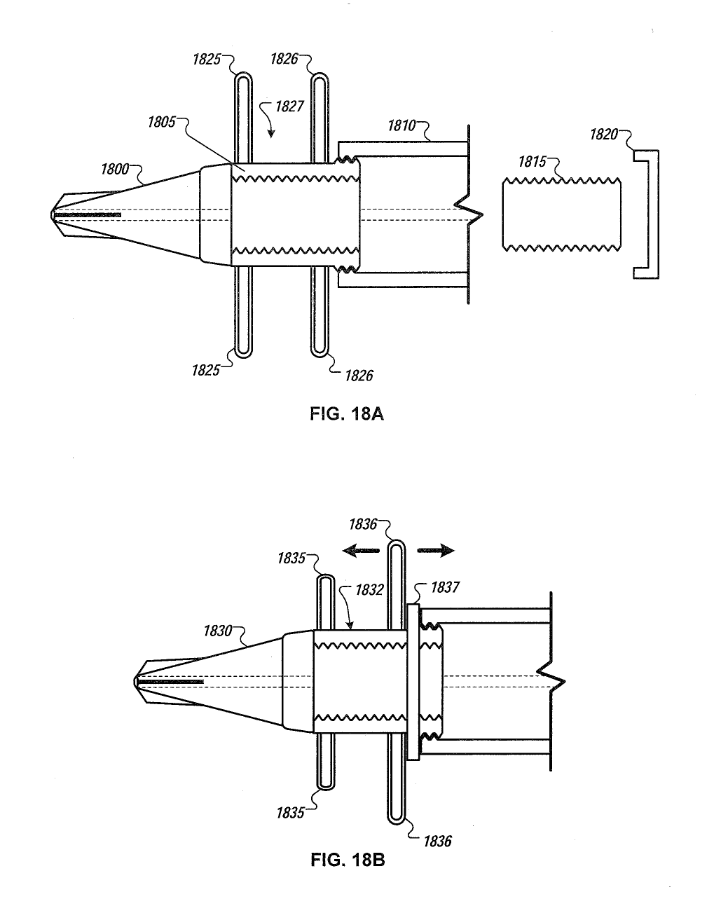

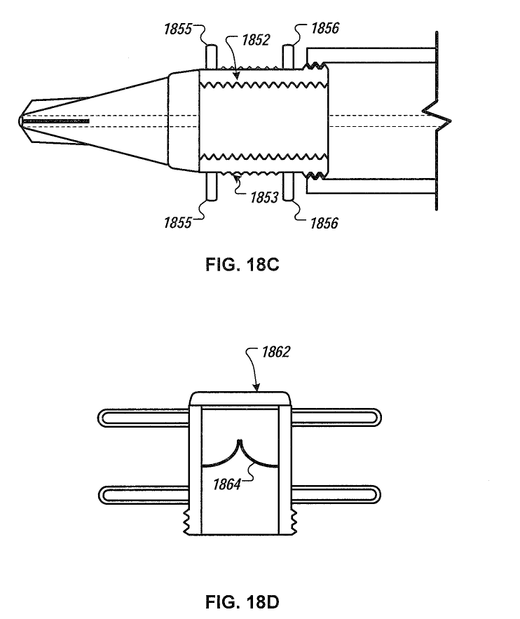

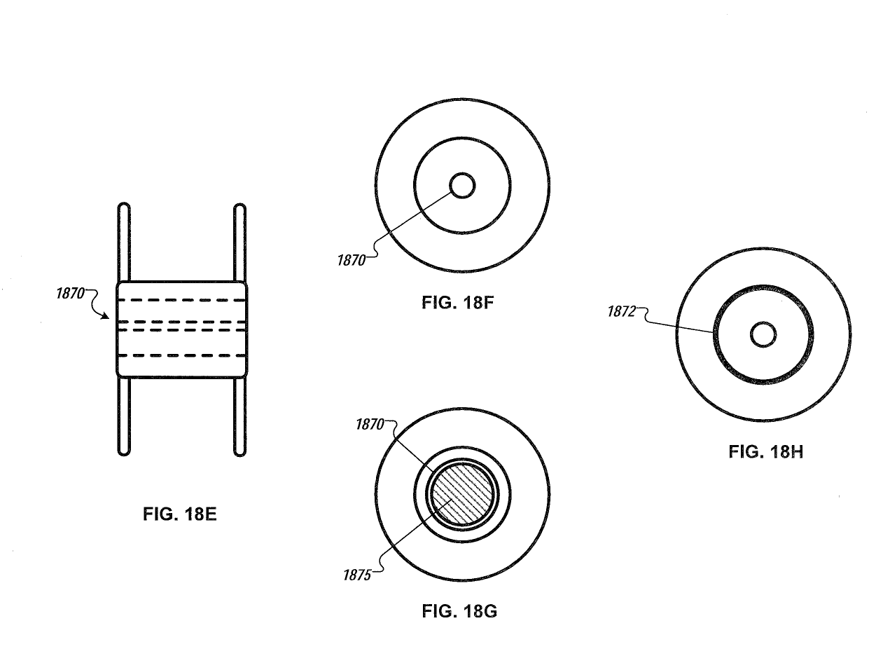

Referring now to FIGS. 18A-18C, there exists a variety of port assembly designs. In some embodiments, a cylindrical port including a central lumen is at least partially surrounded by flexible discs. The discs can include one or more of a flexible polymer (e.g., silicone), a shape memory alloy (e.g., nitinol), and the like. The discs can be made to be compliant, flexible and expandable. The discs can be covered with fabric (Dacron, etc.) or polymers, for example, to facilitate homeostasis. FIG. 18A depicts various elements of the system, including an entry device 1800, a port 1805, a access/delivery sheath 1810, a plug 1815, and a cap 1820. Discs 1825 and 1826 can be held in the collapsed state (not shown) by the delivery sheath 1810 until in positioned in a desired location. As the sheath 1810 is removed, the discs 1825 and 1826 can expand to substantially their full size and facilitate securement of the port 1805. The space 1827 between the discs can be variable to accommodate differing wall thickness. The discs 1825 and 1826 can contain a polymer/drug coating to treat different conditions (anti-thrombolytics, anti-inflammatories, pro-endothelial coatings, collagen, and the like). The discs 1825 and 1826 can be circular but can be other shapes (e.g., oval, elliptical, square, triangular, and the like). The discs 1825 and 1826 can be compliant and, as such, can conform to the shape of the interior of a ventricle or the exterior of a heart. The ventricular (inner) disc 1825 can be slightly convex to conform to the inner curvature of the ventricle and the pericardial (outer) disc 1826 can be slightly concave to conform to the curvature of the exterior surface of the heart. The discs 1825 and 1826 can be flexible so as to lie against the surface of the port 1805 during insertion so as to reduce the diameter of the opening needed in a heart, thereby decreasing the chance of wall tearing.

FIG. 18B depicts a system in which a ventricular (inner) disc 1835 is smaller and made of less material (e.g., nitinol) than a pericardial (outer) disc 1836. The smaller disc 1835 allows for easier placement with less material to push through the wall and therefore reduces the risk of tearing. The pericardial disc 1836 can be adjustable so as to allow the operator to space the discs to the patient's anatomy. The disc 1836 can be pushed with a pusher element along the shaft of the port 1830. Once in position, a locking element 1837 can be slid behind the disc 1836 and locked into position to hold the disc 1836. The locking element 1837 can be configured to secure itself to the port shaft 1832 (e.g., with threads, friction, adhesive, pins, and the like). Both discs 1835 and 1836 can be coated with an echogenic/fluoroscopic coating to help the operator determine via imaging when the discs 1835 and 1836 have the desired spacing, for example, relative to the patient's anatomy.

Referring now to FIG. 18C depicts a configuration with a small silicone ventricular disc 1855 and the option to have no pericardial disc 1856 (although a small silicone pericardial disc 1856 is shown here). In some embodiments, the exterior of the port body 1852 can be grooved with deep, wide grooves 1853 (e.g., in a spiral, and the like) to secure the port body 1852 to the muscle and to provide homeostasis. The grooves 1853 can be embedded with a polymer/drug to facilitate homeostasis (clotting factors) or to promote tissue in-growth (collagen, porous surface, etc.)

Referring now to FIG. 18D, a port 1862 can contain a valve 1864 within its lumen rather than a plug. The “port-valve” 1864 can be placed at the annulus of a cardiac valve and function as a valve replacement. The valve 1864 can be a tissue/polymer valve or a mechanical valve. The port-valve 1864 can be placed at the aortic, pulmonary, mitral, or tricuspid valve sites to replace the native valve.

Referring now to FIGS. 18E-H, in some embodiments, body/wall 1870 of the port lumen could be made of a flexible, shape-memory material (e.g. Nitinol). The port lumen could then exist in a collapsed state (FIG. 18F) until a device (catheter, introducer, etc) is placed into the lumen. The port lumen would then act as an iris and dialate up to accommodate the device (FIG. 18G). Once the device is removed, the lumen would collapse back to the substantially closed state. The port lumen could be configured to include a structure of nitinol weave. The inner lumen of the port lumen can be coated with a material to prevent damage to devices inserted into it (silicone, fabric, etc). FIG. 18F shows the port lumen in the collapsed state, while FIG. 18G shows the port lumen in the open state with a device inserted within it. FIG. 18H shows the option of having a relatively rigid outer lumen wall 1872 (e.g., including plastic, metal, and the like) to hold tissue away from the iris to allow for it to expand easier. The nitinol iris is inside of the rigid lumen wall.

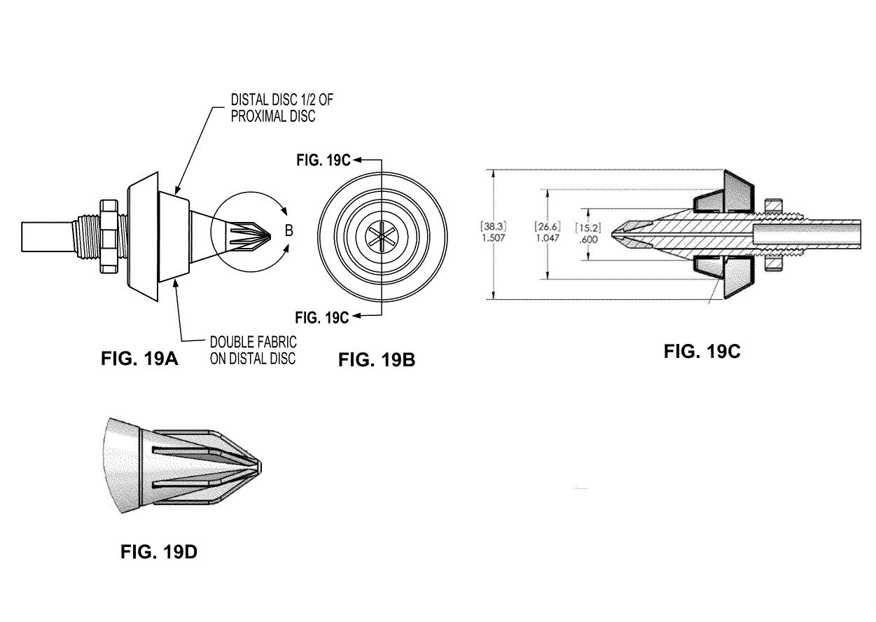

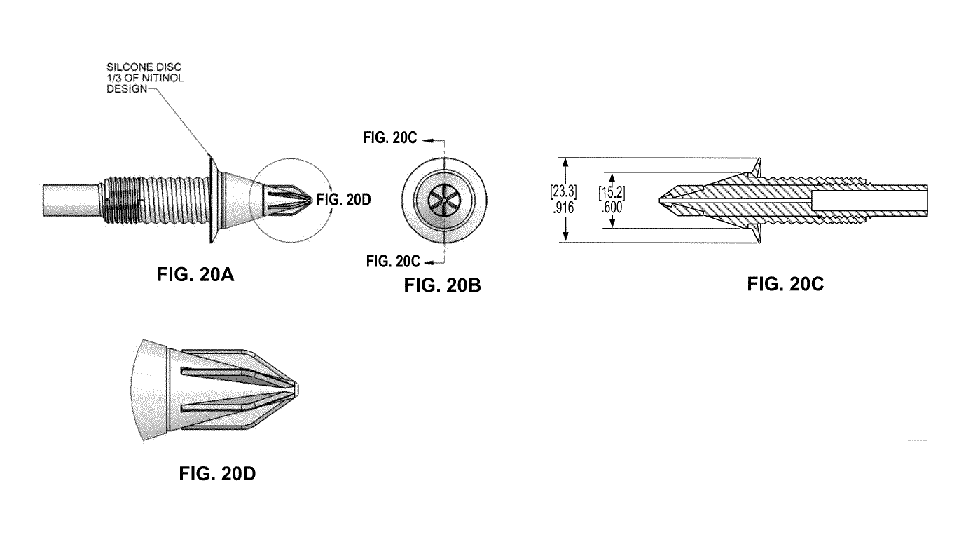

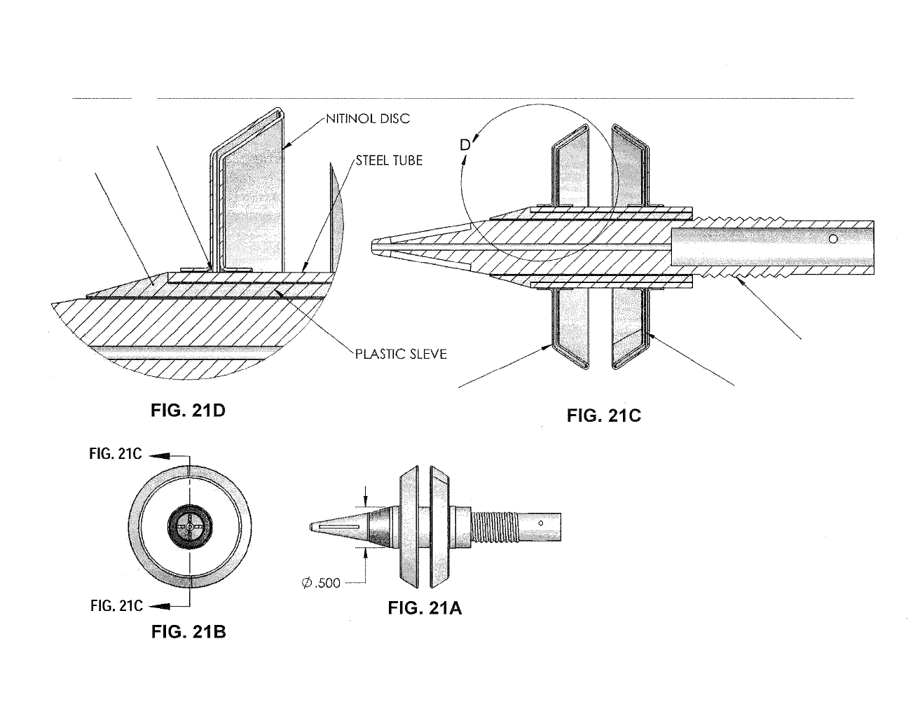

FIGS. 19A-D, 20A-D, and 21A-D depict alternate embodiments of the heart access device and placement system.

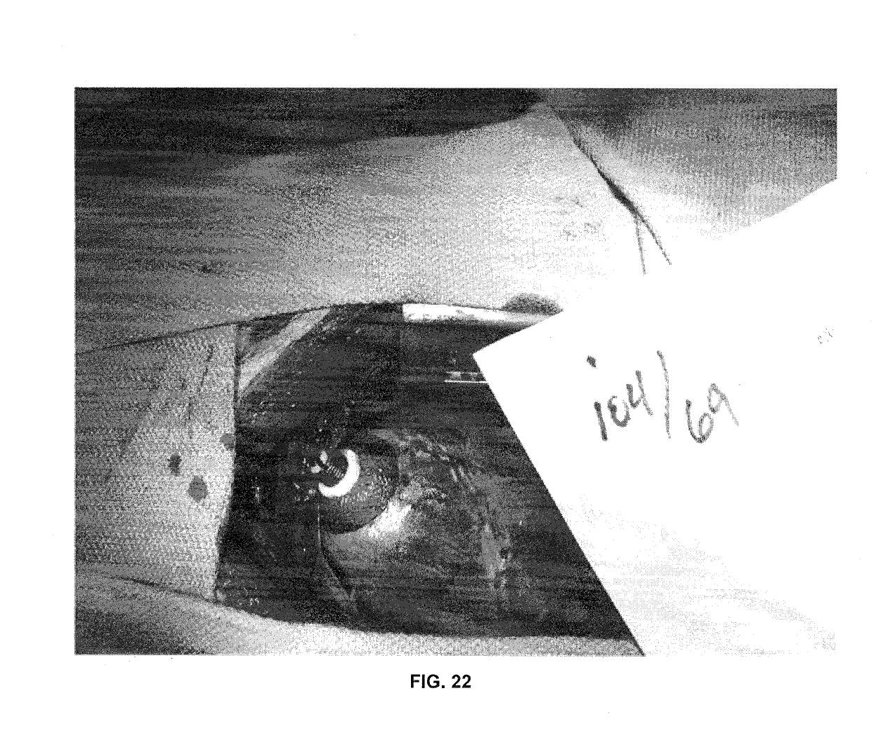

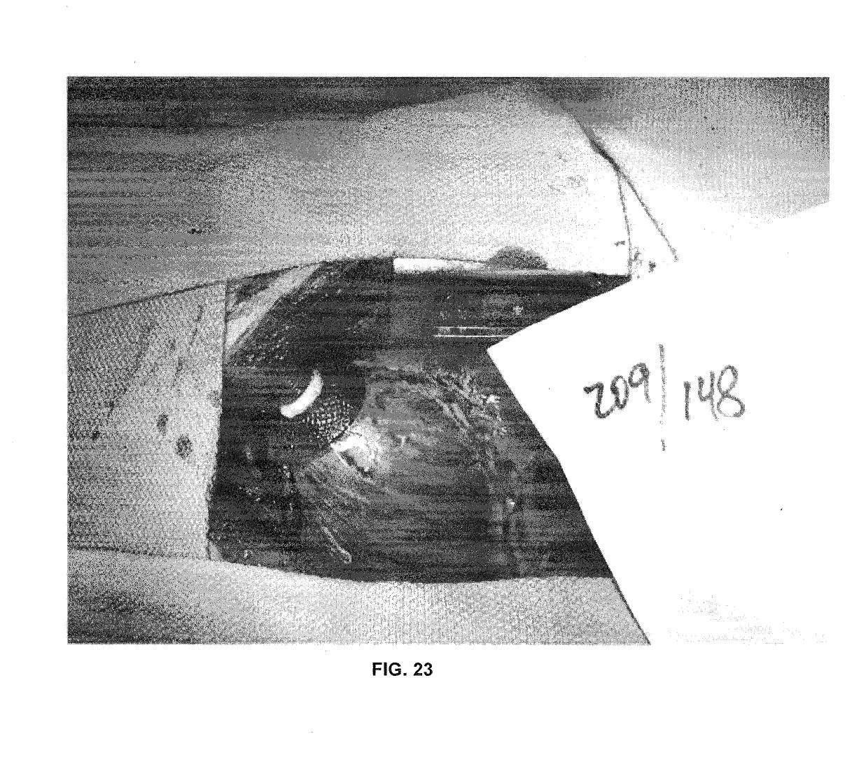

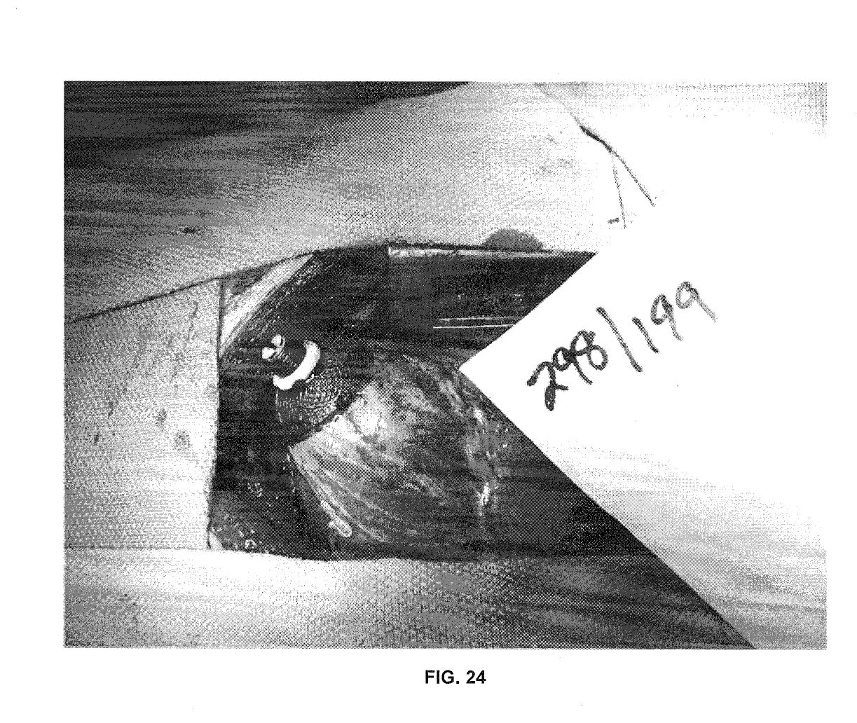

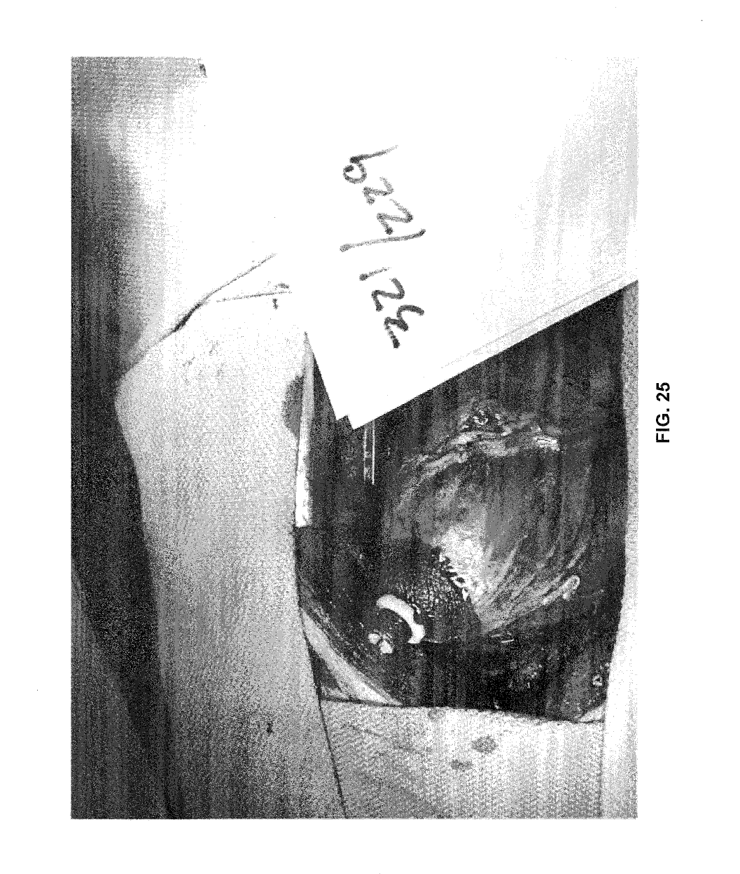

Referring now to FIGS. 22-25, in some embodiments, the system can include features that ease the insertion of a heart access assembly (including a port assembly and entry device) into the wall of a heart easier and with reduced damage to cardiac tissue. For example, the disc material can include a very thin silicone or nitinol layer. In another example, the distal tip of the introducer can include blades. One embodiment of the heart access assembly was tested in an explanted pig heart and then in a live pig. The tested device worked very well once inserted (held pressure both in the explanted heart and in the live animal and in the live pig achieved rapid homeostasis). FIGS. 22-25 show the device implanted in a pig with a blood pressure reading listed on a piece of paper in each figure. Even at high pressures, the device remained in place and did not leak.

It should also be noted that this system can be used to establish port access to other areas of the heart beyond the apex (e.g., trans-septal, trans-atrial, trans-atrial appendage, etc.) and to other organs of the body (e.g., stomach, bladder, bowel, vessels, abdominal cavity, etc.). In particular, the system could be used to facilitate access for Natural Orifice Trans-Endoscopic Surgery (NOTES), which utilizes the stomach/bladder for access to the abdominal cavity to perform a variety of surgical procedures.

Although a few implementations have been described in detail above, other modifications are possible. For example, the logic flows depicted in the figures do not require the particular order shown, or sequential order, to achieve desirable results. In addition, other steps may be provided, or steps may be eliminated, from the described flows, and other components may be added to, or removed from, the described systems and devices. Accordingly, other implementations are within the scope of the following claims.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. (canceled)

2. A transapical heart port system, wherein said system comprises:
(a) a transapical heart port comprising a housing having a first end region and a second end region, wherein said housing defines a channel extending between said first end region and said second end region, wherein said first end region is configured to be inserted into a chamber of a heart of a mammal, wherein said channel is configured to provide repeated access to said chamber through said channel, wherein said first end region comprises a securing portion configured to secure said first end region to an interior region of said heart, wherein said second end region comprises a securing portion configured to secure said second end region to an exterior region of said heart, and wherein said second end region comprises threads for attachment of a sheath and threads for attachment of a plug,
(b) said sheath comprising an end region comprising threads, wherein said sheath is attachable to said transapical heart port via said threads of said end region of said sheath, wherein said sheath, when attached to said transapical heart port, provides access to said second end region through a chest wall covering said heart, and
(c) said plug comprising threads, wherein said plug is attachable to said transapical heart port via said threads of said plug, and wherein at least a portion of said plug, when attached to said transapical heart port, is located within said channel and configured to provide hemostasis.

3. The system of claim 2, wherein said transapical heart port comprises pliable material that allows flexion of said port during beating of said heart.

4. The system of claim 2, wherein said transapical heart port comprises a hemostatic valve attached to said housing and located within said channel.

5. The system of claim 4, wherein said hemostatic valve is configured to reduce blood loss from said heart through said channel when said plug is removed from said transapical heart port.

6. The system of claim 2, wherein said transapical heart port comprises two or more hemostatic valves attached to said housing and located within said channel.

7. The system of claim 2, wherein the size of said channel allows passage of a prosthetic heart valve.

8. The system of claim 2, wherein said securing portion of said first end region or said securing portion of said second end region comprises hooks configured to be embedded within the myocardium of said heart.

9. The system of claim 8, wherein said hooks comprise a shape memory alloy for self-embedding within the myocardium upon deployment of said transapical heart port into said heart.

10. The system of claim 2, wherein said securing portion of said first end region and said securing portion of said second end region comprise hooks configured to be embedded within the myocardium of said heart.

11. The system of claim 10, wherein said hooks comprise a shape memory alloy for self-embedding within the myocardium upon deployment of said transapical heart port into said heart.

12. The system of claim 2, wherein said sheath comprises a hemostatic valve.

13. The system of claim 2, wherein said sheath comprises two or more hemostatic valves.

14. A method for implanting a transapical heart port into a heart of a mammal to provide repeated entry into said heart, wherein said transapical heart port comprises a housing having a first end region and a second end region, wherein said housing defines a channel extending between said first end region and said second end region, wherein said first end region is configured to be inserted into a chamber of said heart, wherein said channel is configured to provide repeated access to said chamber through said channel, wherein said first end region comprises a securing portion configured to secure said first end region to an interior region of said heart, wherein said second end region comprises a securing portion configured to secure said second end region to an exterior region of said heart, and wherein said second end region comprises threads for attachment of a sheath and threads for attachment of a plug,
wherein said method comprises:
(a) positioning said transapical heart port for transapical insertion into said heart, wherein said transapical heart port is attached to said sheath via threads,
(b) inserting said transapical heart port into said heart,
(c) securing said securing portion of said first end region to an interior region of said heart and securing said securing portion of said second end region to an exterior region of said heart,
(d) attaching said plug to said transapical heart port via threads, and
(e) removing said sheath from said transapical heart port.

15. The method of claim 14, wherein said mammal is a human.

16. The method of claim 14, wherein said transapical heart port comprises a hemostatic valve attached to said housing and located within said channel.

17. The method of claim 16, wherein said hemostatic valve is configured to reduce blood loss from said heart through said channel when said plug is removed from said transapical heart port.

18. The method of claim 14, wherein the size of said channel allows passage of a prosthetic heart valve.

19. The method of claim 14, wherein said securing portion of said first end region or said securing portion of said second end region comprises hooks configured to be embedded within the myocardium of said heart.

20. The method of claim 14, wherein said sheath comprises a hemostatic valve.

21. The method of claim 14, wherein said sheath comprises two or more hemostatic valves.
